(19) United States
(12) Patent Application Publication
(10) Pub. No.: US 2011/0034397 A1
(43) Pub. Date: Feb. 10, 2011

(54) INTESTINAL IMMUNE SYSTEM STIMULATOR

(76) Inventors: Tomohiro Kodera, Kawasaki-shi (JP); Noriki Nio, Tokyo (JP); Norimasa Onishi, Tokyo (JP); Yoshinori Mine, Shizuoka-shi (JP)

Correspondence Address: CERMAK NAKAJIMA LLP;ACS LLC, 127 S. Peyton Street, Suite 210, ALEXANDRIA, VA, 22314, US

(21) Appl. No.: 12/856,948

(22) Filed: Aug. 16, 2010

Related U.S. Application Data
(63) Continuation of application No. PCT/JP2009/052462, filed on Feb. 16, 2009.

(30) Foreign Application Priority Data
Feb. 15, 2008 (JP) 2008-034820

(51) Int. Cl. A61K 38/05; A61P 35/00; A61P 37/08; A61P 1/12

(52) U.S. Cl. 514/19.2; 514/21.91

(57) ABSTRACT

Disclosed is an agent, γ-glutamylcysteine, which can effectively and safely stimulate the intestinal immune system. γ-Glutamylcysteine can be formulated in a pharmaceutical composition, a food, and/or a beverage, and used for the treatment or prevention of infectious diseases, diarrhea, polyps, tumors, enteritis or allergy.

This application is a continuation under 35 U.S.C. §120 of PCT Patent Application No. PCT/JP2009/052462, filed Feb. 16, 2009, which claims priority under 35 U.S.C. §119 to Japanese Patent Application No. 2008-034820, filed on Feb. 15, 2008, which are incorporated in their entireties by reference.

### BACKGROUND OF THE INVENTION

1. Field of the Invention

The present invention relates to an agent and compositions containing this agent, which is able to stimulate the intestinal immune system. The agent can be used in pharmaceuticals, foods, and the like.

2. Brief Description of the Related Art

γ-Glutamylcysteine (γ-Glu-Cys, γ-EC) is a dipeptide which is a precursor of glutathione. Foods containing a high level of cysteine can be obtained by the addition of a yeast extract containing γ-glutamylcysteine which has been heated or enzymatically treated (WO 00/30474 A1).

It has also been reported that γ-glutamylcysteine and its derivatives or analogs are able to activate the calcium sensing receptor (CaSR), and further CaSR agonists can be used as therapeutic agents for various diseases (WO 2007/55388 A2).

An anti-rheumatic agent containing a substance which is able to decrease the amount of reduced glutathione in macrophage cells has been reported to suppress production of IL-12, production of NO, and production of INF-γ, resulting in suppression of a delayed hypersensitivity reaction. It has also been reported that compounds such as γ-glutamylcysteine or γ-glutamylcysteine diethyl ester are capable of increasing the amount of reduced glutathione in macrophage cells (JP 2000-309532 A).

Furthermore, an immunomodulator containing a substance which is able to alter the amount of reduced glutathione in macrophage cells has been reported, for example, and the substance includes γ-glutamylcysteine diethyl ester or the like (JP 11-246435 A). In addition, an antidiabetic agent containing a substance which is able to alter the amount of reduced glutathione in macrophage cells has also been reported, and γ-glutamylcysteine and γ-glutamylcysteine dimethyl ester are examples of the substance (JP 2000-309543 A).

An agent for preventing infections containing a combination of cystine, a cysteine compound, or a derivative thereof with theanine has been reported (WO 2003/068214 A1).

However, it has not been previously reported that γ-glutamylcysteine can remarkably stimulate the intestinal immune system. Furthermore, a yeast extract or the like containing γ-glutamylcysteine can be used to manufacture a food containing cysteine has been reported, but a food containing, in a final form, a significant amount of γ-glutamylcysteine has not been previously reported.

### SUMMARY OF THE INVENTION

An aspect of the present invention is to provide an agent which is able to stimulate the intestinal immune system effectively and safely, and compositions containing the agent such as a pharmaceutical, a food, or a beverage. The agent is γ-glutamylcysteine, which has been found to be able to stimulate the intestinal immune system.

It is an aspect of the present invention to provide a composition comprising γ-glutamylcysteine.

It is a further aspect of the present invention to provide the composition as described above, wherein said γ-glutamylcysteine is present in the composition in an amount of 0.000001% or more by mass.

It is a further aspect of the present invention to provide the composition as described above, which is a food or beverage.

It is a further aspect of the present invention to provide the composition as described above, wherein said γ-glutamylcysteine is present in the food or beverage in an amount of 0.000001% or more by mass.

It is a further aspect of the present invention to provide a method for stimulating the intestinal immune system in a subject in need of stimulation of the intestinal immune system comprising administering a composition comprising γ-glutamylcysteine to the subject.

It is a further aspect of the present invention to provide a method for treating or preventing a condition selected from the group consisting of infection, diarrhea, polyp, tumor, enteritis, allergy, and combinations thereof, the method comprising administering a composition comprising γ-glutamylcysteine to a subject having said condition.

It is a further aspect of the present invention to provide the methods as described above, wherein said γ-glutamylcysteine is present in said composition in an amount of 0.000001% or more by mass.

### BRIEF DESCRIPTION OF THE DRAWINGS

FIG. 1 is a graph illustrating the amounts of IgA produced when stimulating mouse spleen cells with LPS. In this graph, EC represents γ-glutamylcysteine, and GSH represents glutathione (the same is true for the following figures).

FIG. 2 is a graph illustrating the amounts of IgG produced when stimulating mouse spleen cells with LPS.

FIG. 3 is a graph illustrating the amounts of IgA produced when stimulating mouse spleen cells with ConA.

FIG. 4 is a graph illustrating the amounts of IgG produced when stimulating mouse spleen cells with ConA.

### DESCRIPTION OF THE PREFERRED EMBODIMENTS

Hereinafter, the present invention is described in detail.

A composition is disclosed which contains an agent which is able to stimulate the intestinal immune system, such as γ-glutamylcysteine. γ-glutamylcysteine can be prepared from, for example, a yeast containing γ-glutamylcysteine.

The yeast containing γ-glutamylcysteine can include a yeast which overexpresses γ-glutamylcysteine synthetase (Otake et al., Bioscience and Industry, 50(10), 989-994, 1992); a yeast in which glutathione synthetase has been modified so that the glutathione synthetase is not able to function by disrupting the glutathione synthetase gene (WO 00/30474 A1); a yeast which harbors glutathione synthetase with an isoleucine residue substituted for the threonine residue at position 47 and an aspartic acid residue substituted for the glycine residue at position 387, and which produces γ-glutamylcysteine (US 2003159049, EP 1449913); a yeast able to produce γ-glutamylcycteine and harboring a mutant MET30 gene which encodes a protein with an amino acid other than serine substituted for the serine at position 569 (JP 2004-113155 A); Saccharomyces cerevisiae in which the C-terminus region from the arginine residue at position 370 of glutathione synthetase gene on the chromosome is deleted (US 2003124684, EP 1201747); and Candida utilis in which the gene encoding glutathione synthetase has been modified to decrease intracellular glutathione synthetase activity (EP 1489173).

Furthermore, methods for obtaining a yeast which is able to produce γ-glutamylcycteine include selecting yeast strains which are resistant to a certain concentration range of MNNG, have attenuated glutathione synthetase activity, and have the ability to produce γ-glutamylcycteine (US 2004214308, EP 1452585); and culturing a yeast which is auxotrophic for pantothenic acid in a medium containing a sufficient amount of pantothenic acid so the yeast can proliferate, and culturing the yeast in a medium having a limited amount of pantothenic acid to increase the content of γ-glutamylcysteine in the yeast cells (JP 2004-201677 A).

If the yeast which produces γ-glutamylcysteine as described above is cultured under suitable conditions, yeast containing γ-glutamylcysteine or a culture containing the same can be obtained.

The medium to be used in the culture of the yeast is not particularly limited as long as the yeast can grow well and produce γ-glutamylcysteine efficiently, and generally, a medium typically used in industry can be used. It should be noted that, if necessary, necessary nutrients can be added to the medium depending on the type of the yeast chosen.

For example, in the case of Saccharomyces cerevisiae, the yeast is cultured to the logarithmic growth phase or stationary phase, and then the culture medium is inoculated into a nutrient medium in an amount of 2%, and the yeast is cultured at 30° C. for 10 to 30 hours.

The yeast culture obtained as above or a fraction thereof contains γ-glutamylcysteine. γ-Glutamylcysteine can be purified, or the yeast culture, yeast cells, a yeast cell homogenate, a yeast cell extract (yeast extract), or the like can be used as long as γ-glutamylcysteine is present. Moreover, a fraction containing γ-glutamylcysteine can be obtained from the yeast cell homogenate or yeast extract.

γ-Glutamylcysteine can be in the form of a salt. The salt may be any salt as long as it is pharmacologically acceptable, and examples include an ammonium salt; a salt with an alkali metal such as sodium or potassium; a salt with an alkaline-earth metal such as calcium or magnesium; an aluminum salt; a zinc salt; a salt with an organic amine such as triethylamine, ethanolamine, morpholine, pyrrolidine, piperidine, piperazine, or dicyclohexylamine; and a salt with a basic amino acid such as arginine or lysine.

The composition in accordance with the presently disclosed subject matter contains γ-glutamylcysteine as an active ingredient. The composition can be formulated as, for example, a pharmaceutical, a quasi drug, or in foods or beverages.

The intestines are known to be the largest immune organ, and overall immunity can be improved by stimulating the intestinal immune system. Therefore, the composition can be effective for the treatment or prevention of diseases which involve the intestinal immune system such as, for example, a variety of infections, diarrhea, polyps, tumors, enteritis, or allergies.

The infection can include viral infections and bacterial infections. The viral infections are not particularly limited, and examples of the viral infections include gastrointestinal virus infections (infections caused by gastrointestinal viruses such as enterovirus and cytomegalovirus), respiratory virus infections (infections caused by respiratory viruses such as influenza virus, rhinovirus, coronavirus, parainfluenza virus, RS virus, adenovirus, and reovirus), herpes zoster caused by herpesvirus, diarrhea caused by rotavirus, viral hepatitis, and AIDS. The composition can be particularly effective for the gastrointestinal virus infections.

Furthermore, the bacterial infections are not particularly limited, and examples include infections caused by Bacillus cereus, Vibrio parahaemolyticus, enterohemorrhagic Escherichia coli, Staphylococcus aureus, MRSA, Salmonella, Clostridium botulinum, and Candida.

The phrase “stimulation of the intestinal immune system” can mean activation of the intestinal immune system which can be independent of other immune system functions. Specifically, for example, the stimulation can mean the promotion of IgA secretion in the intestines, and in particular, in the small intestine.

Depression of the immune system of the intestines can cause diseases, and examples of symptoms/diseases which are consistently related to the depressed immunity of the intestines include infections, allergic diseases, polyps, tumors, and enteritis (Lecture on intestinal immune system, www.ioudou.co.jp/col/archives/2004/11/post_{—}7.html).

Meanwhile, as shown in the examples below, γ-glutamylcysteine promotes IgA secretion in the intestines, and in particular, in the small intestine. Secretory IgA binds specifically to a microorganism such as an invasive bacterium or virus to inhibit adhesion of the microorganism to the epidermal cells. Moreover, the secretory IgA is known to neutralize toxins produced by a bacterium such as Vibrio cholera, and to bind to food-bourne allergens to inhibit absorption of the dietary antigen into the body (Hisako YASUI, Intestinal immune system-regulating action of bifidobacterium, www.healthist.jp/special/150_{—}03/03_{—}03.html).

Moreover, secretory IgA is known to be involved in the induction of “oral immune tolerance”, which is an immunosuppressive mechanism, for a protein absorbed from the intestines (Satoshi HACHIMURA, Intestinal immune system as contact between food and immune system: Unique cell responsiveness, jsbba.bt.a.u-tokyo.ac.jp/03reikai3/hachimura.pdf). Allergic reactions can be suppressed by induction of oral immune tolerance.

Therefore, stimulation of the intestinal immune system, in particular, the promotion of IgA secretion in the intestines can be effective for the treatment or prevention of the above-described symptoms/diseases.

Methods of administration of the composition containing the agent which can stimulate the intestinal immune system are not particularly limited, and can include oral administration, invasive administration, such as via injection or the like, suppository administration, or transdermal administration. The composition can include a solid or liquid non-toxic pharmaceutical carrier suitable for the chosen administration method. The composition can be formulated in any appropriate form, and can include a solid such as a tablet, a granule, a powder, or a capsule; a liquid such as a solution, a suspension, or an emulsion; and can be freeze-dried. The formulation can be prepared by a conventional method.

Examples of the above-mentioned non-toxic pharmaceutical carrier include glucose, lactose, sucrose, starch, mannitol, dextrin, fatty acid glyceride, polyethylene glycol, hydroxyethyl starch, ethylene glycol, polyoxyethylene sorbitan fatty acid ester, gelatin, albumin, amino acids, water, and physiological saline. Furthermore, if necessary, a commonly used additive such as a stabilizer, a humectant, an emulsifier, a binder, or a tonicity agent can also be appropriately added.

The composition, in addition to the active ingredient γ-glutamylcysteine, can also contain one or more other medicaments effective for treatment of the target disease, such as a substance which is able to stimulate the intestinal immune system or is able to treat or prevent infections. Examples of such medicaments include cystine or a derivative thereof, theanine, and lactic acid bacteria.

The dosage or intake of the composition containing γ-glutamylcysteine can be an amount effective for the treatment or prevention, and is appropriately adjusted depending on the patient's age, sex, weight, symptom, etc. For example, in the case of oral administration, the amount of γ-glutamylcysteine per dose can be 0.0005 g to 1 g per kg body weight, or in another example, 0.001 g to 0.2 g per kg body weight. The number of administrations per day is not particularly limited, and administration can be performed once to several times a day.

The amount of γ-glutamylcysteine in the composition is not particularly limited as long as it is suitable for the above-described dosage. The amount is preferably 0.000001% to 70% by mass, or in another example, 0.00001% to 50% by mass, or in another example, 0.0001% to 40% by mass, per dry weight.

The food or beverage can contain γ-glutamylcysteine. The kind of the food or beverage is not particularly limited, and examples of the food or beverage include seasonings, a fermented food, an alcoholic beverage, soup, sauce, mayonnaise, dressing, curry roux, juice, a nutritional beverage, rice gruel, bread, confectionery, a retort pouch food, a frozen food, a supplement, a milk product, and a cosmetic food.

The food or beverage can be manufactured by the same method using the same materials as is typical or normal for the manufacture the food or beverage, except that the food or beverage contains γ-glutamylcysteine. The materials are not particularly limited, and examples of the materials can include: rice, barley, and corn starch for an alcohol beverage; flour, sugar, salt, butter, and fermentation yeast for bread; and soybean and wheat for a fermented food.

The food or beverage can contain γ-glutamylcysteine in an amount of 0.000001% by mass or more, or in another example, 0.01% by mass or more, or in another example 0.05% by mass or more, or in another example 0.1% by mass or more. The upper limit of the amount of γ-glutamylcysteine is not particularly limited but is preferably 50% by mass or less, or in another example 10% by mass or less, or in another example 5% by mass or less.

The food or beverage can stimulate the intestinal immune system, and for example, the container or package thereof can state that the food or beverage has the stimulatory effect on the intestinal immune system, or that the food or beverage has a therapeutic or preventive effect for the above-described diseases.

### EXAMPLES

Hereinafter, the present invention is described more specifically by way of the following non-limiting examples. The γ-glutamylcysteine used in the examples was obtained by custom synthesis and purification by PEPTIDE INSTITUTE, INC.

### Example 1

Six- to eight-week-old BALB mice (female) were fed for 1 week a commercially-available solid feed and provided with tap water. To evaluate the influence of γ-glutamylcysteine on antibody production, a culture system using mouse spleen cells was employed. The spleen cells were collected from the BALB-c mice (n=3), then washed and suspended in RPMI 1640 medium (10% FCS, 50 U/ml penicillin, 50 μg/ml streptomycin). The cells were adjusted so that there were 1×10⁶ cells per 48-well culture plate, and then either γ-glutamylcysteine or glutathione (purchased from Sigma) each dissolved in a phosphate buffer, was added to the plates. A phosphate buffer was added to plates as a control. Then, the cells were stimulated with 5 μg of LPS (lipopolysaccharide, obtained from Sigma).

The cells were again adjusted so that there were 1×10⁶ cells per 48-well culture plate, and then stimulated with 0.5 μg of ConA (concanavalin A) in the presence or absence of γ-glutamylcysteine or glutathione. The cells were then cultured in a CO₂ incubator at 37° C. for 5 days, and the amounts of the antibodies in the supernatant were analyzed.

The amounts of IgA and IgG were measured by the ELISA method. The method for measuring IgG is described below. 100 μl of a rat anti-mouse IgG antibody (Calbiochem, 1 μg/ml 50 mM sodium carbonate buffer, pH 8.5) solution was added to a 96-well ELISA plate, and the plate was incubated at 4° C. overnight to coat each well with the anti-mouse IgG antibody. The plate was washed with PBST (phosphate buffered saline, 0.05% Tween20) three times and then subjected to a blocking treatment with 200 μl of a PBS solution containing 2% BSA at 37° C. for 1 hr.

The plate was washed with PBST three times, and 100 μl of the culture supernatant (diluted 9-fold with PBST containing 1% BSA) was added, followed by a reaction at 37° C. for 2 hours. The plate was washed again with PBST four times. 100 μl of an alkaline phosphatase-conjugated anti-mouse IgG (rabbit) (BD Biosciences) solution diluted 2000-fold with PBST containing 1% BSA was added, followed by a reaction at 37° C. for 1 hour. The plate was washed six times, and then color was developed with p-nitrophenyl phosphate. The reaction was stopped with 100 μl of 3 M NaOH/well, and the absorbance was measured at 405 nm.

IgA was measured in accordance with the same method as for IgG except that a rat anti-mouse IgA antibody (BD Biosciences) was used as the immobilized antibody. Also, a biotinylated anti-mouse IgA antibody (rat) (BD Biosciences) was used as the labeling antibody, a 3,3′,5,5′-tetramethylbenzidine solution (TMB substrate-developing solution, Sigma) was used as the chromogenic substrate, color development was stopped with 1 M sulfuric acid, and the absorbance was measured at 450 nm.

Statistical processing was performed by the Student's t-test, and a P value of 5% or less was considered significant.

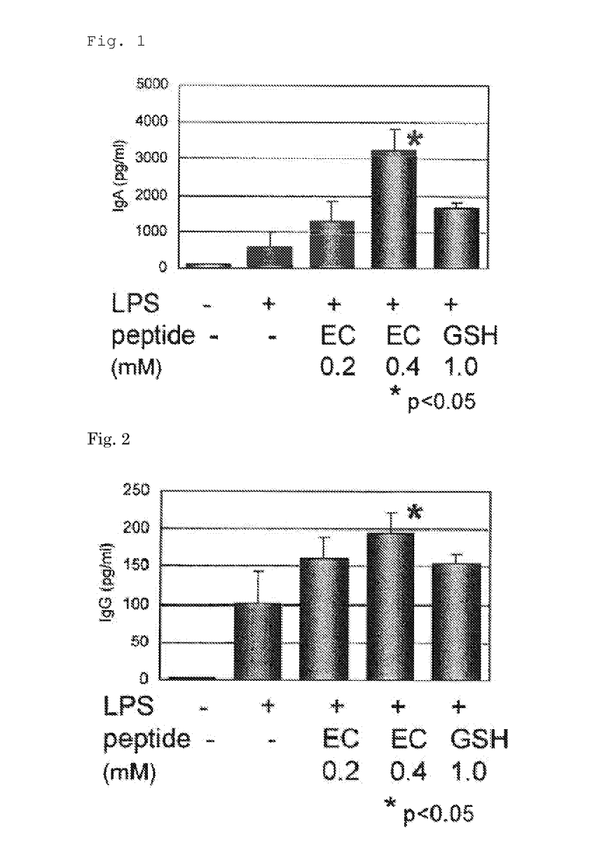

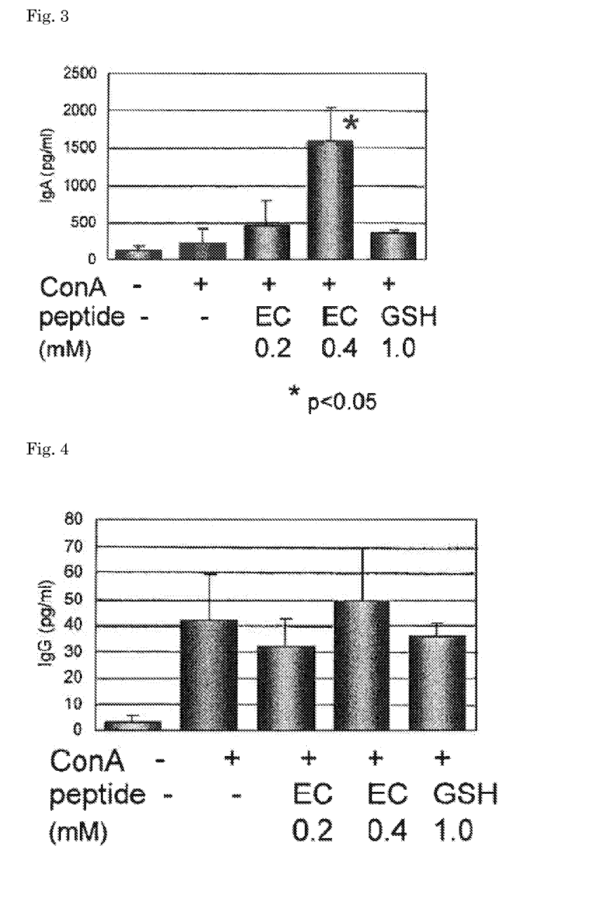

The results are shown in FIGS. 1 to 4. For both ConA and LPS stimulation, the peptides were able to significantly increase the production of IgA as compared to control. Alternatively, the ability to increase production of IgG was lower than for IgA. From this data, γ-glutamylcysteine appears to be able to better increase the production of IgA. In addition, γ-glutamylcysteine appears to be able to increase production of IgA as compared with glutathione.

### Example 2

Five-week-old BALB/c male mice (n=6) were fed for 1 week and then used in this experiment. A commercially-available feed was used, and the mice' access to the feed was unrestricted, and tap water was provided. γ-Glutamylcysteine was dissolved in sterilized physiological saline, and gavage administration was performed at 10 mg/kg B.W./day. The administration period was 7 days. During the administration period, the body weights were measured every day immediately before the administration to determine the dosage. There was no difference between the body weights of the different groups.

On day 7 after the start of the test, a necropsy examination was performed. Anesthesia was performed by interperitoneal administration with a pentobarbital formulation. At laparotomy, blood was collected from the abdominal aorta. The small intestinal contents were also collected.

Serum was collected from the blood, and the total IgA and total IgG concentrations were measured using an ELISA kit. The intestinal contents were diluted 10-fold with PBS and suspended sufficiently, and centrifugation was then performed to separate the serum. The total IgA concentration was measured by ELISA in the same way as in Example 1.

The results are shown in Table 1. In the table, γ-EC represents γ-glutamylcysteine. The table shows relative indices of the respective test groups compared to the control group to which γ-glutamylcysteine was not administered, which was defined as 1. The amount of IgA present in the small intestine was found to increase upon ingestion of the peptide. However, the amounts of serum IgA and serum IgG were found not to increase, and it was confirmed that the peptide was able to stimulate the immunoactivity in the intestinal mucosa.

TABLE 1
Antibody-producing activity of peptide

| Item | Non-administered group | γ-EC administered group |
|---|---|---|
| Small intestine IgA | 1.0 | 3.0 |
| Serum IgA | 1.0 | 0.3 |
| Serum IgG | 1.0 | 0.6 |

Relative indices with respect to the value of the non-administered control group defined as 1

### INDUSTRIAL APPLICABILITY

The intestinal immune system stimulator of the present invention can stimulate the intestinal immune system safely and effectively. Further, the food or beverage of the present invention has an action of stimulating the intestinal immune system.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A method for stimulating the intestinal immune system in a subject in need of stimulation of the intestinal immune system comprising administering a composition comprising γ-glutamylcysteine to the subject.

2. The method of claim 1, wherein said γ-glutamylcysteine is present in the composition in an amount of 0.000001% or more by mass.

3. A method for treating or preventing a condition selected from the group consisting of an infection, diarrhea, polyp, tumor, enteritis, allergy, and combinations thereof, said method comprising administering a composition comprising γ-glutamylcysteine to a subject having said condition.

4. The method of claim 3, wherein said γ-glutamylcysteine is present in said composition in an amount of 0.000001% or more by mass.
